(19) United States
(12) Patent Application Publication
(10) Pub. No.: US 2022/0287690 A1
(43) Pub. Date: Sep. 15, 2022

(54) BIOLOGICAL SAMPLE COLLECTION DEVICE AND METHOD OF USING SAME

(71) Applicant: THE ROYAL INSTITUTION FOR THE ADVANCEMENT OF LEARNING/MCGILL UNIVERSITY, Montréal (CA)

(72) Inventors: Lucy GILBERT, Westmount (CA); Rosaire MONGRAIN, Montreal (CA); Ramses GALAZ, Col. Banus (MX)

(21) Appl. No.: 17/636,478

(22) PCT Filed: Aug. 14, 2020
(86) PCT No.: PCT/IB2020/057686

§ 371 Date: Feb. 18, 2022

Related U.S. Application Data
(60) Provisional application No. 62/889,175, filed on Aug. 20, 2019.

(51) Int. Cl. A61B 10/00; A61B 10/02

(52) U.S. Cl. CPC A61B 10/007; A61B 10/0283; A61B 2010/0216

(57) ABSTRACT

A method of collecting a biological sample inside a body cavity (18) using filaments (132). The body cavity (18) is accessed with the filaments (132) contained in a sheath (126) with ends thereof spaced apart from each other. To collect the sample, the filaments (132) are deployed outside the sheath (126) in the body cavity (18) and the ends of the filaments (126) are longitudinally brought closer to each other to bulge the portion extending therebetween to a radius larger than the radius of the sheath 126). Also, a device (100, 300) for performing the method.

### FIELD OF THE INVENTION

The present invention relates to general field of biological sample collection. More specifically, the present invention is concerned with intrauterine sample collection devices and methods of using same.

### BACKGROUND

Endometrial and Ovarian cancers are most of the time diagnosed only when apparent symptoms lead a patient to consult a gynecologist. By that time, the cancer has most of the time entered a late stage and may prove to be difficult or impossible to eradicate. Screening patients regularly using intrauterine samples while the cancer is microscopic would help in early diagnosis. Microscopic cancers can be identified by using highly sensitive genomic technology that can identify small amount of cancer DNA among thousands of normal cells. Whole preserved cells and surrounding stroma are not required. However, currently available sample collectors are geared towards collecting material for cytologic or histologic diagnosis. Cytology and histologic testing require tissue that preserves the architectural structure of the cells. The process of dislodging adherent tissue to collect such samples creates trauma and sets off bleeding, which is uncomfortable or painful to the patient. Moreover, the presence of blood and normal tissue reduces the sensitivity of the test. Cancer cells lose their adhesive properties and are more likely to be loose and free in the uterine cavity. To detect small cancers of the uterus or ovaries require samplers that preferentially collect already free cells in the uterine cavity or cells weakly bound to the endometrium, rather than dislodge healthy cells from the endometrial lining.

Against this background, there exists a need in the industry to provide novel intrauterine sample collection devices and methods that collect fluid, loose and superficial cells from the uterine cavity. An object of the present invention is therefore to provide such improved devices and methods.

### SUMMARY OF THE INVENTION

In a broad aspect, there is provided a device for collecting a biological sample, the device comprising: a substantially elongated core including a core first member and a core second member, the core first and second members being longitudinally movable relative to each other, the core further including a plurality of elongated filaments, the filaments each defining longitudinally spaced apart first and second mounting locations, each filament being mounted to the core first and second members respectively at the first and second mounting locations, the filaments each defining a filament detached section between the first and second mounting locations, the filament detached section being deformable and movable relative to the core first and second members; and a substantially elongated sheath receiving at least part of the core thereinto, the sheath defining longitudinally opposed sheath proximal and distal ends, the sheath and the core first and second members being longitudinally movable relative to each other; the device being configurable between a device retracted configuration and a device expanded configuration, wherein, in the device retracted configuration, the filament detached sections are contained within the sheath, and, in the device expanded configuration, at least part of the filament detached sections is distally outside the sheath; the core first and second elements being longitudinally movable relative to each other between core stowage and collection configurations, wherein the first and second mounting locations are closer to each other in the core collection configuration than in the core stowage configuration.

There may also be provided a device wherein, in the core stowage configuration, the filament detached sections extend generally longitudinally so as to span a stowage volume extending radially less than the sheath, and, in the core collection configuration, with the device in the device expanded configuration, the filament detached sections span a collection volume extending radially more than the sheath.

There may also be provided a device wherein the collection volume is substantially ellipsoidal.

There may also be provided a device wherein the collection volume is substantially tulip flower shaped.

There may also be provided a device further comprising an actuator for selectively configuring the device between the device retracted and expanded configurations and selectively moving the core between the core stowage and collection configurations.

There may also be provided a device wherein movements between the device retracted and expanded configurations and movements between the core stowage and collection configurations are independent from each other.

There may also be provided a device wherein movements between the device retracted and expanded configurations and movements between the core stowage and collection configurations are linked such that when the device in the device retracted configuration, the core is in the core stowage configuration, and when the device is in the device expanded configuration, the core is in the core collection configuration.

There may also be provided a device wherein the core second member is tubular and surrounds at least part of the core first element, the core first member protruding from the core second member in the core stowage configuration.

There may also be provided a device, wherein the actuator defines an actuator body and first and second sliders movable longitudinally along the actuator body, the first slider being jointly movable with a first element selected from the core first and second members and the sheath, the second slider being jointly movable with a second element different from the first element and selected from the core first and second members and the sheath, and a third element different from the first and second elements and selected from the core first and second members and the sheath being fixed relative to the actuator body.

There may also be provided a device wherein the actuator body is hollow and the first and second sliders are mounted in the actuator body so as to be movable longitudinally relative thereto.

There may also be provided a device wherein the first and second sliders protrude from the actuator body and are independently movable therealong.

There may also be provided a device wherein at least one of the first and second sliders protrudes from the actuator body, the first and second sliders being operatively coupled to each other so that proximally directed and distally directed movements of the first slider occur jointly and simultaneously with respectively distally and proximally directed movements of the second slider, the sheath being movable jointly with the first slider, the core second member being movable jointly with the second slider and the core first member being longitudinally fixed relative to the actuator body.

There may also be provided a device wherein the first and second sliders include respectively first and second racks oriented substantially longitudinally and a pinion extending between the first and second racks and coupling movements of the first and second racks to each other so that the first and second racks move in opposite directions relative to each other.

There may also be provided a device wherein the sheath is in fluid communication with a syringe for allowing exertion of an underpressure or an overpressure relative to atmospheric pressure at the sheath distal end.

There may also be provided a device wherein the filaments are hydrophilic.

There may also be provided a device wherein at least one of the core first member, core second members and sheath is hollow, in fluid communication with a vacuum source and provided with at least one aperture leading thereinto and provided adjacent the filaments, whereby exerting a vacuum using the vacuum source collects fluids adjacent the filaments through the at least one aperture.

In another broad aspect, there is provided a method for collecting a biological sample in a body cavity accessible through a body passageway narrower than the body cavity, the method using an elongated tubular sheath defining a sheath distal end, the method also using a plurality of elongated filaments each defining opposed filament end portions and a filament intermediate portion extending therebetween, the method comprising: with the filaments contained in the sheath with the filament end portions longitudinally spaced apart from each other, inserting the sheath in the body passageway so that the sheath distal end is positioned adjacent to or inside the body cavity; distally deploying the filaments outside the sheath in the body cavity and moving the filament end portions substantially longitudinally towards each other to cause the filaments to buckle in the filament intermediate portions and span a volume extending radially to a greater extent than the sheath; collecting the biological sample with the filaments intermediate portions; withdrawing the filaments inside the sheath; withdrawing the sheath from the body passageway.

There may also be provided a method wherein the biological sample includes biological fluids.

There may also be provided a method wherein the biological sample includes cells.

There may also be provided a method wherein collecting the biological sample includes moving the filament intermediate portions along a mucosa delimiting the body cavity with at least part of the filament intermediate portion and the mucosa parallel to each other.

There may also be provided a method wherein the body cavity is a uterine cavity defined by an uterine wall, collecting the biological sample including moving the filament intermediate portions tangentially along the uterine wall.

Advantageously, in some embodiments, the proposed device is relatively atraumatic and causes minimal discomfort to the patient in use. This allows its use in asymptomatic patients for screening purposes, for example. Additionally, the proposed device is, in some embodiments, relatively inexpensive to manufacture and relatively easy to use in an ergonomic manner.

The present application claims priority from U.S. Provisional patent applications 62/889,175 filed Aug. 20, 2019, the contents of which is hereby incorporated by reference in its entirety.

Other objects, advantages and features of the present invention will become more apparent upon reading of the following non-restrictive description of preferred embodiments thereof, given by way of example only with reference to the accompanying drawings.

### BRIEF DESCRIPTION FOR DRAWINGS

In the appended drawings:

FIG. 1, in a side view, illustrates an embodiment of a device for collecting an intrauterine sample, here shown in a step of a method of using the device;

FIG. 2A, in a side view, illustrates a proximal part of the device of FIG. 1 in a device retracted configuration;

FIG. 2B, in a side view, illustrates a distal part of the device of FIG. 1 in the device retracted configuration;

FIG. 3A, in a side view, illustrates a proximal part of the device of FIG. 1 in a device expanded configuration;

FIG. 3B, in a side view, illustrates a distal part of the device of FIG. 1 in the device expanded configuration;

FIG. 4A, in a side view, illustrates a proximal part of the device of FIG. 1 in a device intermediate configuration;

FIG. 4B, in a side view, illustrates a distal part of the device of FIG. 1 in the device intermediate configuration;

FIG. 5, in a side view, illustrates an other step in the use of the device of FIGS. 1 to 4B;

FIG. 6, in a side view, illustrates yet an other step in the use of the device of FIGS. 1 to 4B;

FIG. 7, in a partial perspective view, illustrates a kit including a container, a lid part and the device of FIGS. 1 to 4B;

FIG. 8, in a side cross-sectional view, illustrates the kit of FIG. 7;

FIG. 9A, in a side view, illustrates an embodiment of a brush bristle part of the device of FIGS. 1 to 4B;

FIG. 9B, in a side view, illustrates an alternative embodiment of a brush bristle part of the device of FIGS. 1 to 4B;

FIG. 9C, in a side view, illustrates an other alternative embodiment of a brush bristle part of the device of FIGS. 1 to 4B;

FIG. 10A, in a side view, illustrates yet an other alternative embodiment of a brush bristle part of the device of FIGS. 1 to 4B;

FIG. 10B, in a side view, illustrates yet an other embodiment of a brush bristle part of the device of FIGS. 1 to 4B;

FIG. 11A, in a transversal cross-sectional view, illustrates yet an other alternative embodiment of a brush bristle part of the device of FIGS. 1 to 4B;

FIG. 11B, in a transversal cross-sectional view, illustrates yet an other alternative embodiment of a brush bristle part of the device of FIGS. 1 to 4B;

FIG. 11C, in a transversal cross-sectional view, illustrates yet an other alternative embodiment of a brush bristle part of the device of FIGS. 1 to 4B;

FIG. 11D, in a transversal cross-sectional view, illustrates yet an other alternative embodiment of a brush bristle part of the device of FIGS. 1 to 4B;

FIG. 11E, in a transversal cross-sectional view, illustrates yet an other alternative embodiment of a brush bristle part of the device of FIGS. 1 to 4B;

FIG. 11F, in a transversal cross-sectional view, illustrates yet an other alternative embodiment of a brush bristle part of the device of FIGS. 1 to 4B;

FIG. 12, in a side cross-sectional view, illustrates a distal end of an alternative device for collecting a biological sample;

FIG. 13A, in a side elevation view with portions removed, illustrates a step in the use of the device of FIG. 12;

FIG. 13B, in a side elevation view with portions removed, illustrates another step in the use of a device similar to the device of FIG. 12;

FIG. 13C, in a side elevation view with portions removed, illustrates yet another step in the use of the device similar to the device of FIG. 12;

FIG. 13D, in a side elevation view with portions removed, illustrates yet another step in the use of the device similar to the device of FIG. 12;

FIG. 13E, in a side elevation view with portions removed, illustrates yet another step in the use of the device of FIG. 12;

FIG. 14, in a perspective view with parts removed, illustrates the device of FIG. 12;

FIG. 15, in a perspective cut away view, illustrates an actuator part of the device of FIG. 12;

FIGS. 16A to 16E, in a perspective view with parts removed, illustrates successive steps in use of the device of FIG. 12;

FIG. 17, in a perspective partially exploded view, illustrates of another alternative device for collecting a biological sample, the device being shown in a device retracted configuration;

FIG. 18, in a perspective partially exploded view, illustrates the device of FIG. 17 shown in a device extended configuration;

FIG. 19, in a perspective cutaway view, illustrates an actuator part of the device of FIGS. 17 and 18; and

FIG. 20, in a side cross-section view, illustrates the actuator of FIG. 19.

### DETAILED DESCRIPTION

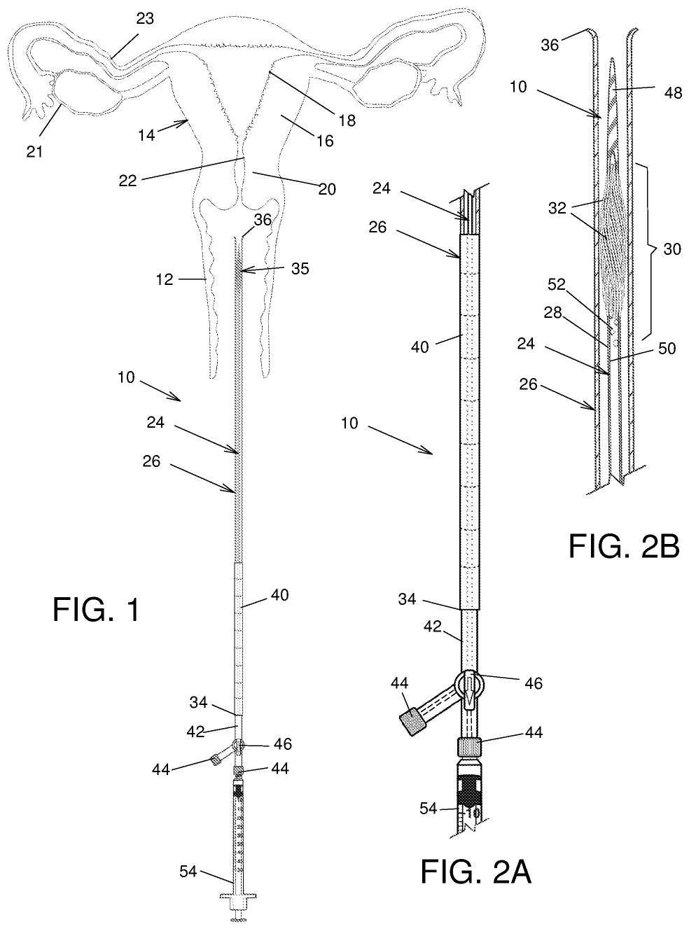

With reference to FIG. 1, there is shown a device 10 for collecting a biological sample from a patient having a vagina 12 leading to a uterus 14 having an uterine wall 16 delimiting a uterine cavity 18 through a cervix 20 having a cervical canal 22. The device 10 is here shown inserted in the vagina 12, prior to actual insertion of part thereof in the uterine cavity 18. The device 10 includes a substantially elongated core 24 and a substantially elongated sheath 26 receiving at least part of the core 24 thereinto and defining axially opposed sheath proximal and distal ends 34 and 36. As seen for example in FIG. 2B, the core 24 defines a core outer surface 28. The core 24 also defines a distal brush section 30. The brush section 30 is provided with brush bristles 32 extending from the core outer surface 28.

In the present document, the terminology distal and proximal refers to the location relative to a physician (not shown in the drawings) using the device 10. Distal elements are closer to the uterine cavity 18 in use, while proximal elements are closer to the physician. Also, the terminology “substantially” and “about” is used to denote variations in the thus qualified terms that have no significant effect on the principle of operation of the device 10. These variations may be minor variations in design or variations due to mechanical tolerances in manufacturing and use of the device 10. These variations are to be seen with the eye of the reader skilled in the art.

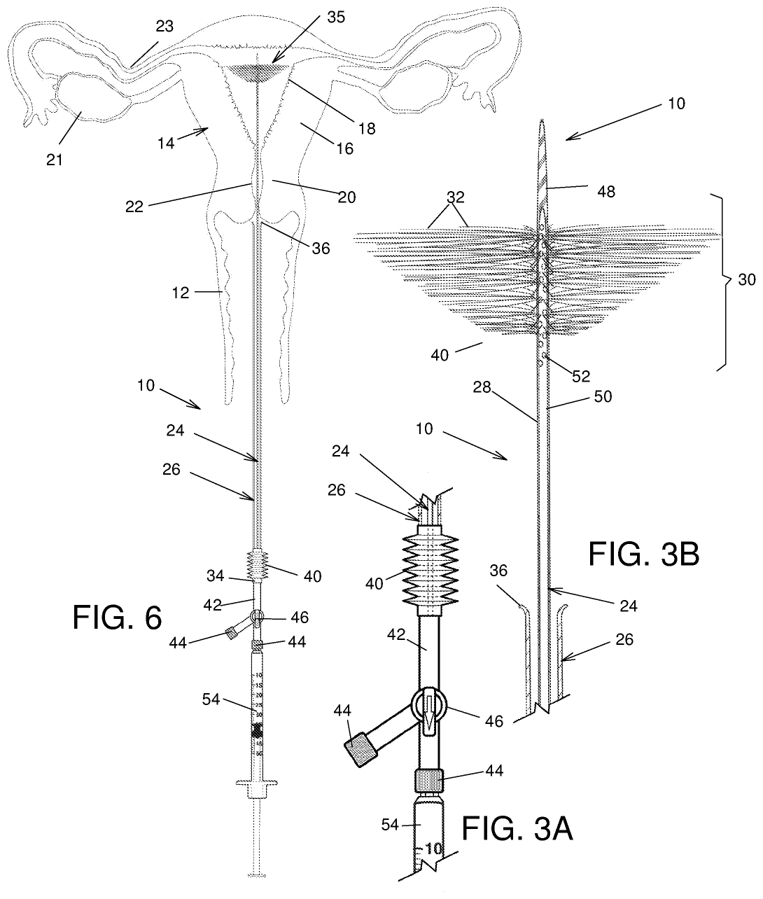

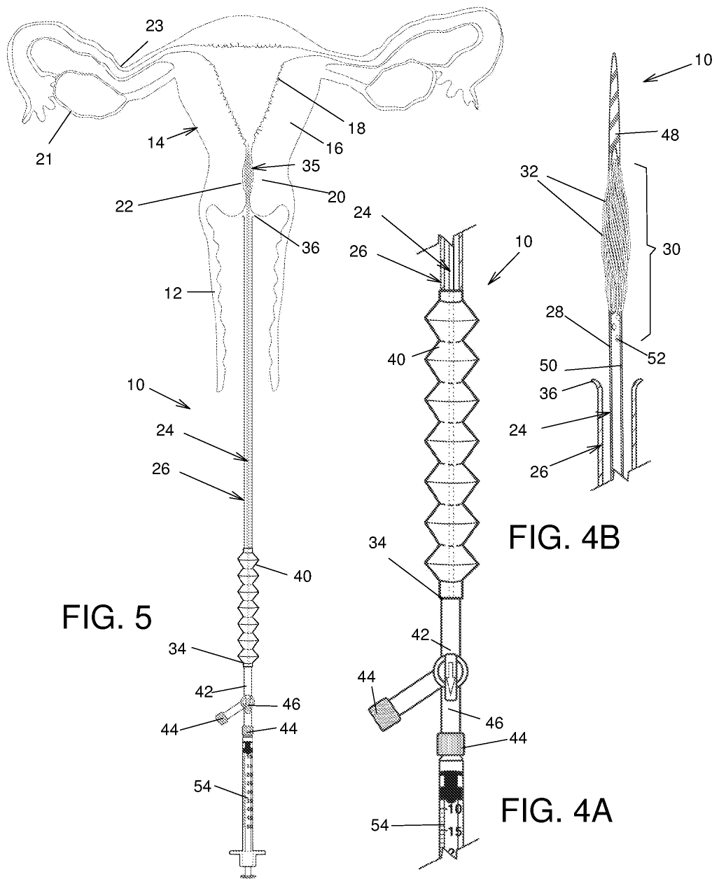

The core 24 and sheath 26 are movable relative to each other between a device retracted configuration (seen in FIGS. 1, 2A and 2B) and a device expanded configuration (seen in FIGS. 6, 3A and 3B). FIGS. 5, 4A and 4B illustrate the device 10 in a device intermediate configuration, achieved when transitioning between the device expanded and retracted configurations. Referring to FIG. 2B, in the device retracted configuration, the brush bristles 32 are contained within the sheath 26, for example compressively, in a brush bristle compressed configuration. Referring to FIG. 3B, in the device expanded configuration, the brush bristles 32 are outside the sheath 26, distally to the sheath distal end 36, in a brush bristle expanded configuration wherein the brush bristles 32 span a larger volume than in brush bristle compressed configuration.

The sheath 26 is substantially elongated and typically tubular. In some embodiments, the sheath 26 is flexible at least in its distal portion, along with a corresponding portion of the core 24, to accommodate in use the typically curved shape of the human vagina 12. In some embodiments, the sheath 26 is substantially chalice-shaped and firm at the sheath distal end 36. In other words, the sheath expands in diameter at the sheath distal end 36 and presents a smooth curved surface to the cervix 20 to improve patient comfort. The wider sheath distal end 36 helps in collecting any adjacent leaked intrauterine fluids.

While the core 24 and the sheath 26 could in some embodiments be of constant length and simply axially slidable relative to each other, in other embodiments, as shown in the drawings, the sheath 26 includes an axially collapsible section 40 movable between collapsible section expanded and retracted configuration, shown respectively in FIGS. 2A and 3A. The sheath 26 is shorter in the collapsible section retracted configuration than in the collapsible section expanded configuration. For example, the axially collapsible section 40 includes a flexible section of the sheath 26 that includes preformed folds allowing the axially collapsible section 40 to retract and expand in an accordion-like manner.

In some embodiments, the core 24 and the sheath 26 are both proximally fixedly mounted to a base 42 so that in the collapsible section retracted configuration, the core 24 and sheath 26 are in the device expanded configuration, and in the collapsible section expanded configuration, the core 24 and sheath 26 are in the device retracted configuration. In such embodiments, insertion and withdrawal of the device 10 in the vagina 12 is decoupled from the expansion of the device 10 to expose the brush bristles 32 and retraction of the device 10 to withdraw the brush bristles 32 in the sheath 26, which facilitates use of the device 10. The base 42 is typically substantially rigid and may then serve as a handle for the device 10.

In some embodiments, the base 42 is hollow and includes at least one Luer connector 44, the purpose of which is described in further details hereinbelow. The base 42 may include two or more Luer connectors 44 selectable for fluid communication with the remainder of the device 10 using a valve 46.

With reference to FIG. 6, in a specific embodiment, the brush bristles 32 are configured and sized so that in the brush bristle expanded configuration, the brush bristles 32 conform to a shape of at least part of the uterine cavity 18 so that the brush bristles 32 contact the uterine wall 16 along a circumference thereof. It may be the case that the brush bristle expanded configuration, the brush bristles 32 can only conform to part of the uterine cavity 18, as shown in FIG. 6 where the brush would conform to the portion of the uterine cavity 18 that is adjacent the cervix 20. In other embodiments, the brush bristles 32 are sized to contact the uterine wall 16 along most of the circumference volume spanned by the brush bristles 32. It should be notes that FIG. 6 is schematic in that in many women, the uterus 14 is collapsed and does not define a large uterine cavity 18 as shown in FIG. 6.

In such configuration, the brush bristles 32 can therefore easily contact a large surface area of the uterine wall 16 to facilitate collection of a relatively large number of cells therefrom. This is to be contrasted to, for example, to the endometrial pipelle used for biopsies, which is much smaller than the uterine cavity 18. The large contact area between the brush bristles 32 and the uterine wall 16 helps in sampling simultaneously cells from diverse portions of the endometrium. Also, this large contact area allows collection of a relatively large number of cells without inflicting trauma to the endometrium. Yet furthermore, since the brush bristles 32 span a large portion of the uterine cavity 18 volume, cells floating in intrauterine fluids can more easily contact the brush bristles 32 for collection. In some embodiments, the brush bristles have a composition such that cells relatively easily stick to them, or at least stick preferentially to them relative to free floating in intrauterine fluids.

In some embodiments, in the brush bristle expanded configuration, the brush bristles 32 span a volume that has at least a portion thereof that tapers proximally. For example, the brush bristles 32 span a volume that entirely tapers proximally. A non-limiting example of such a volume is a substantially frusto-conical shape. It should be noted that in other embodiments, the brush bristles 32 span any other suitable volume.

In some embodiments, initially, prior to insertion in the uterus 14, the brush bristles 32 extend proximally from the core outer surface 28 to facilitate insertion in the cervical canal 22. In some embodiments, in the brush bristle expanded configuration, the brush bristles 32 expand substantially radially outwardly from the core outer surface 28.

In a very specific and non-limiting example, the frusto-conical shape has an opening angle of between about 60 degrees and about 120 degrees, the brush bristles have a length comprised between a minimal length and a maximal length, the minimal length being between about 2 mm and about 5 mm and the maximal length being between about 10 mm and about 25 mm and the brush bristles extend from a section of the core that is about 10 to 25 mm long.

The brush bristles 32 are typically relatively flexible to minimize discomfort to the patient and reduce or eliminate the need for anaesthesia, as opposed to many current cell collection methods. In a typical embodiment, the brush bristles 32 have a stiffness (flexural strength) that is small to enable collection of cells without excessively traumatizing the uterine wall 16, but that nevertheless have the ability to engage the uterine wall 16 with enough friction to dislodge individual cells for collection. It should be noted that this is in contract with biopsy devices that need to be much more rigid as such devices intend to remove tissue samples, while preserving the tissue structure, which is not the case necessarily in the present invention. Therefore, stiffness is such that the brush bristles 32 are stiff enough to collect cells while not significantly exfoliating the uterine wall 16. The brush bristles 32 may be made of a polymer, such as non-limitingly polyvinyl chloride (PVC), polypropylene (PP), polyethylene terephthalate (PET), Nylon, polyvinylidene fluoride (PVDF), low-density polyethylene (LDPE), high-density polyethylene (HDPE), or ultra-high-molecular-weight polyethylene (UHMWPE), among others, or a metal, such as non-limitingly a Ni-Ti superelastic metal. In some embodiments, the brush bristles 32 are non-DNA containing and are therefore made of materials that are not plant or animal based. In some embodiments, the brush bristles 32 are configured and sized and have material properties such that the brush bristles 32 exert a maximum shear stress of 10 Pa or less on uterine tissues in use. In some embodiments, the brush bristles 32 are configured and sized and have material properties such that the brush bristles 32 exert a maximum shear force of 1 N or less on uterine tissues in use.

In some embodiments, the brush bristles 32 are attached to the core 24 by any assembly method such as adhesive bonding, ultrasonic welding, sintering, laser welding, radio-frequency bonding, mechanical lock or interference, among other possibilities. In other embodiments, the brush bristles 32 are manufactured using laser-cut methods from a polymer or metal tube. Other suitable manufacturing methods are also possible.

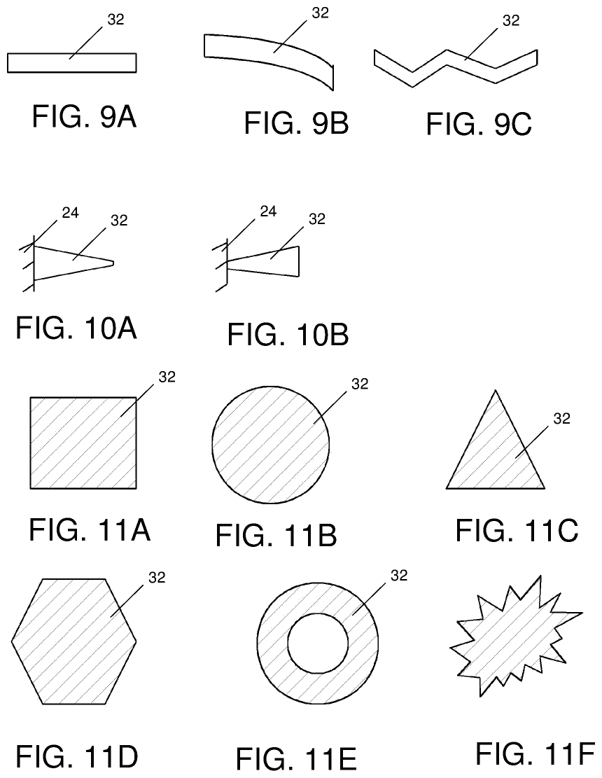

The brush bristles 32 may be in any suitable number and may have any suitable shape. All the brush bristles 32 may have the same general configuration, or brush bristles 32 of different configurations may be mixed together in the same device 10. For example, as seen respectively in FIGS. 9A, 9B and 9C, the brush bristles 32 may be respectively substantially rectilinear, substantially curved or substantially jagged in the expanded configuration, among other possibilities. Also, in another example, as seen respectively in FIGS. 9A, 10A and 10B, the brush bristles 32 may be respectively of constant diameter, taper in a direction leading away from the core 24 or taper in a direction leading towards the core 24, among other possibilities. In yet another example, as seen respectively in FIGS. 11A to 11F, the brush bristles 32 may have a transversal cross-sectional configuration selected from the group consisting of a square, a round, a triangular, a polygonal, a ring and an irregular configuration among other possibilities.

In some embodiments (not shown in the drawings), the brush section 30 terminates distally the core 24. However, as seen for example in FIG. 2B, in other embodiments, the core 24 further defines a tip section 48 distal to the brush section 30. For example, and non-limitingly, the tip section 48 is about 0.5 cm to 1.5 cm long and has a maximal diameter of about 1.5 to 2.5 mm, to terminate at a tip having a diameter of about 0.5 to 1 mm. For example, the tip section 48 is atraumatic and reduces discomfort and injury risk when the core 24 is inserted in the cervical canal 22. For example, in some embodiments, the tip section 48 has a diameter smaller than a diameter of the core 24 in the brush section 30. Therefore, in opposition to some existing devices, such as endometrial pipelles, the tip of the core 24 is not larger than other portions of the core. Typically, the tip section 48 tapers in a distally leading direction, and has for example and non-limitingly a substantially conical shape with a rounded tip. In some embodiments, the tip section 48 is more flexible than the brush section 30.

Still referring to FIG. 2B, in some embodiments, the core 24 defines an inner passageway 50 extending axially therealong and at least one aperture 52 extending between the core outer surface 28 and the inner passageway 50 in the brush section 30. Typically, a plurality of such apertures 52 are provided, which extend for example substantially radially. Having the apertures 52 in the brush section 30 ensure that the apertures 52 are spaced apart from the uterine wall 16, described below, which would be uncomfortable to the patient and could cause tissue damage. Therefore, the bush bristles 32 also act as spacers for spacing apart the apertures 52 from the uterine wall 16.

The inner passageway 50 is proximally in fluid communication with a vacuum device usable to create a pressure drop in the inner passageway 50. In a specific embodiment of the invention, the vacuum device takes the form of a syringe 54, secured to one of the Luer connectors 44. In such embodiments, the base 42 is hollow to provide a communication between the syringe 54 and the inner passageway 50. It should be noted that other vacuum devices could be used, such as a pump, among other possibilities.

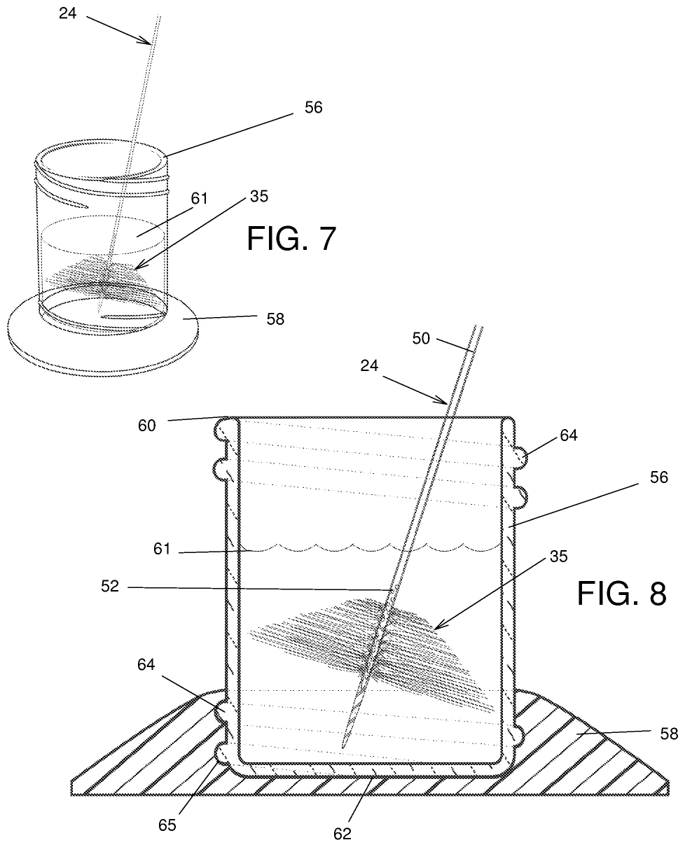

With reference to FIGS. 7 and 8, in some embodiments, the device 10 is usable with a container 56 and a lid 58. For example, the container 56 contains about 4 ml of a cell preserving fluid, but other quantities are within the scope of the invention. A non-limiting example of a suitable cell preserving fluid is a genomic DNA preserving buffer solution. The container 56 is typically deep enough to ensure the whole length of the tip section 48 and brush section 30 is submerged in the buffer.

In some embodiments, the lid 58 is selectively screwable to the container 56 at a top end 60 thereof to close the container 56 and at a bottom end 62 thereof to provide a base for supporting the container 56, the lid 58 being wider than the container 56. To that effect, the container 56 is for example substantially cylindrical and provided with external threads 64 both at the top end 60 and at the bottom end 62. The lid 58 is for example frusto-conical and provided with internal threads 65 that are configured to engage the external threads 64. The container 56 and lid 58 may conform to any suitable standard in the industry for fluid containers intended for shipping. A suitable box and label (not shown in the drawings) may be also provided so that once the container 56 has received biological material from the device 10, as described below, the closed container 56 can be shipped to laboratory for sample analysis. In some embodiments, the device 10, or portions thereof, is provided sterilized in a sealed envelope.

The core 24 and sheath 26 are typically made of any suitable medical grade material, such as plastic, polymer, or similar material. The core 24 may have for example, and non-limitingly, a maximal diameter of from about 1.5 to about 2.5 mm.

An example of a method of using the device 10 is now described referring to the sequence of FIGS. 1, 5, 6 and 7. The brush section 30, along with the brush bristles 32, are collectively referred to as a brush 35 hereinbelow. This method may also be performed using other devices similar to the device 10, but that are not identical thereto. The method is used to collect a biological sample from the uterus 14. This biological sample may include cells and, in some embodiments, intrauterine fluids. The intrauterine fluids may include cells in suspension or freely floating generic material, among other possibilities. It should be noted that while obviously uterine cells, such as endometrial cells may be collected, ovarian cells that have travelled from the ovaries 21 to the uterus 14 through the fallopian tubes 23 may also be collected in some embodiments.

The patient is typically placed in the lithotomy position, further to which a sterile, lightly lubricated vaginal speculum is employed to render the external os of the uterus 14 visible. If needed, the cervix 20 may be steadied with a tenaculum. To minimize contamination risks and maximize cell collection, in some embodiments, the biological sample is to be taken before any other intrauterine intervention. Referring to FIG. 1, with the brush 35 retracted in the sheath 26, the sheath 26 is then inserted into the vagina 12 until the sheath 26 abuts against the cervix 20. Then, as seen in FIG. 5, while keeping the sheath 26 abutted against the cervix 20, the brush 35 is pushed out of the sheath 26 so that the brush 35 enters the cervical canal 22 and then the uterine cavity 18, as seen in FIG. 6. Then, the brush 35 may conform to the shape of at least a portion of the uterine cavity 18 as the brush bristles 32 are resiliently deployed. This deployment is performed by collapsing the collapsible section 40, which effectively shortens the sheath 26 to expose the brush bristles 32. When present, the tip section 48 facilitates penetration in the cervical canal 22. Afterwards, the brush 35 is rotated axially to collect uterine cells on the brush 35, for example and non-limitingly about 360 degrees, and the brush 35 is withdrawn back into the sheath 26 so that the sheath 26 can be withdrawn from the vagina 12 while protecting the brush 35 from contamination. When the brush 35 is withdrawn back into the sheath 26, the brush bristles 32 extend distally from the core 24, as opposed to proximally prior to insertion.

When the container 56 is provided, after having withdrawn the sheath from the vagina, the brush 35 is again pushed out of the sheath 26 and the brush 35 is plunged in the collection fluid 61 to collect at least part of the uterine cells deposited on the brush bristles 32. In alternative embodiments, the brush 35 may be severed from the remainder of the core 24 and plunged in the collection fluid 61. Severance may be performed by simply cutting the brush 35 with scissors, or the brush 35 may be detached from the remainder of the core 24 by breaking a preformed weakened section of the core (not shown in the drawings).

When inner passageway 50 and syringe 54 are present, the method may also include, when the brush 35 is deployed in the uterus 14, aspiring intrauterine fluids in the inner passageway 50 through the apertures 52 by creating a suction with the syringe 54. For example, the intrauterine fluids are undiluted biological fluids. Vacuum is maintained afterwards until the intrauterine fluids may be collected in the collection fluid 61 by pressing the syringe 54 plunger. In another example, the second Luer connector 44 is used to attach a sterile fluid source thereto to provide a wash to assist in collection of the cells and intrauterine fluids. Then, the method includes pushing a sterile fluid, such as a sterile saline solution, in the uterus 14, and withdrawing, the injected fluid using the syringe 54. In such methods, the intrauterine fluids or collected sterile fluid may also be transferred to the collection fluid 61.

In some embodiments, the above collection methods allow collection of enough genetic material to diagnose cancer. In some embodiments, distinction between benign somatic mutations and malignant somatic mutations may be made. Therefore, there may be provided a method of diagnosing cancer in a patient, the method comprising collecting a biological sample as described hereinabove using the device 10 and collecting a germ line sample from the patient. Then, the method includes using next-generation sequencing (NGS) methods on the germ line sample and the biological sample to identify somatic mutations. Finally, the method includes diagnosing the patient as having cancer or as having only benign mutations on a basis of the somatic mutations. For example, the germ line sample includes blood cells. In these embodiments, collecting the germ line sample includes drawing a blood sample from the patient. In another example, the germ line sample includes buccal endothelial cells. In these embodiments, collecting the germ line sample includes performing a buccal swab or having the patient spit a saliva sample. Any other suitable germ line sample may also be used. An example of suitable genetic methods for identifying cancer is described below, but any other suitable genetic method may be used. For example, PCT application publication WO2017220782 published Dec. 28, 2017, which is hereby incorporated by reference in its entirety, describes such a method.

More specifically, as cancer cells exfoliates more easily than normal cells, and as the uterus is a continuous tract from the fallopian tube, a cytologic sample taken from the uterus is likely to have traces of cancer cells very early in the process of carcinogenesis of the ovary, fallopian tube and endometrium. In very early stage, when the tumour is very small, the biological sample will have only a very low number of cancer cells amidst large numbers of normal endometrial cells, precluding pathologic detection. To identify these small number of cancer cells, a DovEEgene™—HaloPlex^{HS}™ System may be used. This system uses next-generation sequencing (NGS) of a genomic segment at very deep coverage to identify cancer mutations in a small fraction of DNA templates from cancer cells and a DNA-tagging error reducing technology to minimize errors resulting from possible PCR amplification bias and confidently identify rare variants in the <1% range.

In summary, this method attempts to identify all subtypes (type I and II) of ovarian and endometrial cancers, at an early stage, using an innovative uterine sampling system (the device 10) as well as a bespoke assay that includes gene panel design, bioinformatic analysis process and machine learning, to identify small amounts of cancer DNA in samples from inside the uterus and distinguish it from non-malignant conditions. In parallel the approach introduces the novelty of being able to identify predisposing germline mutations in the established inherited breast and ovarian cancer genes, BRCA1 and BRCA2, as well as in the predisposing genes PTEN, TP53, MLH1 and MSH2 AKT1, APC, CDKN2A, CTNNB1, FBXW7, FGFR2, KRAS, NRAS, PIK3CA, PIK3R1, PPP2R1A, RNF43 or any other gene of diagnostic value, while also simultaneously detecting somatic mutations in these genes that can identify prevalent cancer and inform treatment decisions. In other embodiments, the genes of interest are ARID1A, AKT1, APC, BRCA1, BRCA2, CDKN2A, CTNNB1, FBXW7, FGFR2, KRAS, MLH1, MSH2, NRAS, PIK3CA, PIK3R1, PPP2R1A, PTEN, RNF43, TP53, EGFR, POLE, MAPK1, BRAF, NF1, RB1, GABRA6, CSMD3, ARID5B, FAT3 and CDK12.

A high sensitivity assay has been developed to detect low frequency mutations at high sensitivity from cells collected with the uterine brush. This assay interrogates the entire coding sequence in contrast to so called “hotspots”. This increases the sensitivity in detecting somatic mutations, and covers genes for which there are no established hotspots and detects germline variants at the same time in genes such as BRCA1 and BRCA2, MLH1, MSH2 genes. The DOvEEgene™-HaloPlex™ assay is based on Agilent DOvEEgene-Haloplex technology and is specially designed to be suitable for all types of samples including formaldehyde-fixed paraffin-embedded (FFPE) tissues. The panel was designed to amplify the entire exonic regions of genes known to be mutated in ovarian cancer, for example is a specific embodiment: AKT1, APC, CDKN2A, CTNNB1, FBXW7, FGFR2, KRAS, NRAS, PIK3CA, PIK3R1, PPP2R1A, PTEN, RNF43, BRCA1, BRCA2, MLH1, MSH2 and TP53 (see FIG. 3 for a description of the design). In total, a few thousand amplicons may be used. Genetic sequencing may for example be performed as follows:

### Preparation of the Samples

1 DNA extraction from saliva samples
Upon reception of saliva samples in an Oragene™ saliva collection kit, genomic DNA (gDNA) is extracted a Chemagen™ MSMI (Perkin-Elmer)

2 DNA extraction from brush samples
Upon reception of brush samples in ThinPrep™ solution, or in genomic DNA preserving buffer, genomic DNA (gDNA) is extracted using a Chemagen™ MSMI (Perkin-Elmer)

3 DNA quantification
Quantification is performed with PicoGreen™ using a Janus liquid handler and a Tecan Spark 10M plate reader

4 DNA integrity verification
Sample integrity and fragment length is verified by running samples on a 1% precast gel

### Sample normalization for subsequent HaloPlex^{HS} capture

5 Normalization of saliva and brush samples
gDNA concentrations between 50 and 550 ng are required. Samples are thus either diluted using the JANUS Varispan Automation Workstation™ (PerkinElmer) or concentrated by hand using NucleoMago™ NGS Clean-up and Size Select beads, based on their concentration as measured previously with PicoGreen™.

### Automated Sample Capture using HaloPlee^{HS}™ on the NGS BRAVO™ Workstation

6 Samples are captured using the HaloPlex^{HS} according to a customized protocol for automisation on the NGS BRAVO workstation (see Annex for further details).
6.1. The normalized gDNA from the previous steps are digested with restriction enzymes.
6.2. The digested DNA is hybridized to the HaloPlex^{HS} probe library.
6.3. The circularized DNA hybrids are purified and ligated.
6.4. The target DNA is captured and washed.
6.5. The captured target library is amplified by PCR.
6.6. The amplified target DNA is purified.
6.7. The enriched target DNA is validated and quantified.
6.8. Validated and quantified samples are pooled for multiplexed sequencing.

### Sequencing on the Illumina HiSeq 2500

7 Pooled samples are sequenced using 100 bp or 125 bp paired-end reads according to Agilent guidelines for HaloPlex^{HS}.

In brief, the targeted capture method is specifically designed to identify low allele frequency variants through the attachment of a 10 nucleotide-long molecular barcode to the captured sample DNA molecules. Typical sensitivity achieved is in the region of 0.4-0.5%, but can detect variants below 0.1%. Confidence in identifying a mutation correctly can be achieved by designing different but overlapping “probes” that are used to capture and analyze different DNA strands that include the DNA sequence of interest. In addition, these probes are designed to be able to target complementary DNA strands marked as “sense” or “antisense”, with both strands being captured in a very large fraction (for example over 99%) of the target. This allows a novel way to analyze the data: during downstream analysis of the sequencing data, molecular barcode sequence data are used to collapse reads originating from the same sample molecule, but also by sequencing the same base from complementary DNA molecules, which improves base calling accuracy by removing artifacts and allows for accurate quantification of the mutant allele fraction within each sample.

After sequencing, various bioinformatics methods may be used. For example, an analytical pipeline that is designed to combine sensitivity with specificity in order to achieve the aims of the assay. Data analysis is carried out using a bespoke pipeline utilizing initially the SureCall™ software tool (Agilent), followed by the following analysis approach. Amplicon probe identification that captures a specific fragment is used, and as we know which strand is targeted by an amplicon probe, we bioinformatically identify which DNA strand is captured and sequenced, marking it as being originally a “sense” or “antisense” DNA strand. If both sense and antisense DNA strand derived sequences agree, then the result is retained. This process is achieved by counting the number of sense and antisense strands that contained each mutation found. This information is then used to either retain or filter away mutations for increasing the specificity of the data produced.

One problem with high sensitivity sequencing is the issue of cross sample contamination, either during sample handling or during the sequencing process within the instrument, as for cost reasons, Next generation sequencing libraries need to be combined in a “multiplexed library” and sequenced as part of one sequencing reaction. The sequencing reads obtained are then assigned to the specific original library through a process involving the analysis of sample specific index sequence. However, minute contaminations are possible. Therefore, a customized variant call filtering approach that is specific for this approach may be used. It involves creating a panel of normal, germline samples which are sequenced at high coverage to create a list of germline variants for all samples to be studied. By identifying mutations that are present in the somatic sample, but not in the germ line sample, various somatic mutation parameters may be identified.

Classification of patients as having cancer or only benign mutation can be done with reference to a reference database, in which the cancer status of the patients are known, by using classification techniques. Parameters used for such classification may include, age, body mass index (BMI), total mutation burden, and presence of specific mutations, among others.

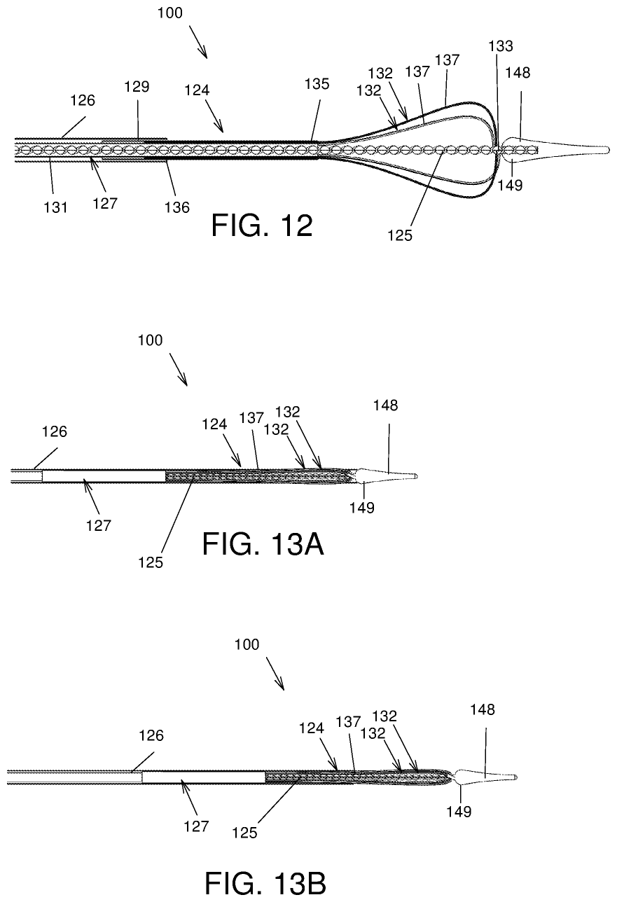

Referring to FIG. 12, there is shown part of an alternative device 100 for collecting a biological sample, for example an intrauterine sample usable in the above-described method. The device 100 may also be adapted in alternative embodiments to collect cells from other body cavities, such as for example the stomach or the intestinal tract, among other possibilities. The device 100 includes a sheath 126, similar to the sheath 26, and an alternative substantially elongated core 124, the sheath 126 receiving at least part of the core 124 thereinto. The sheath 126 is illustrated as having a non-flared distal end, but flared distal ends, as in the sheath 26, are also within the scope of the invention. More generally, when applicable, the various characteristics, options and alternatives of the device 10 are also usable in the device 100.

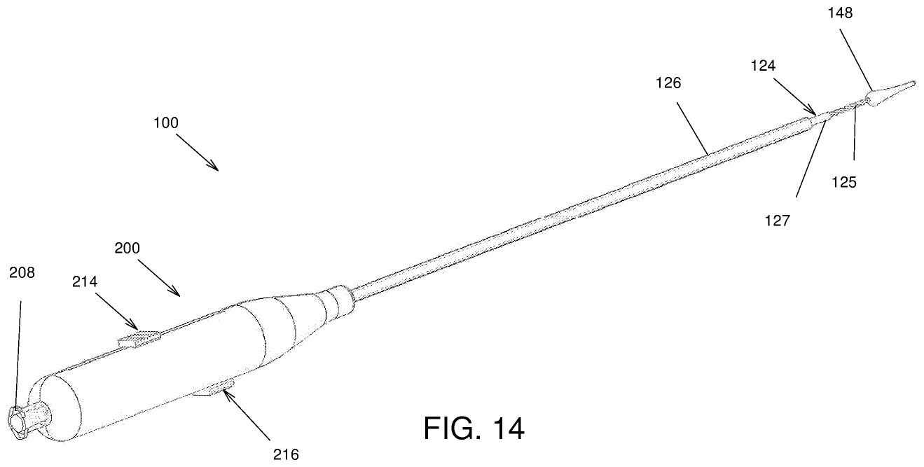

The core 124 includes a core first member 125 and a core second member 127, the core first and second members 125 and 127 being longitudinally movable relative to each other and relative to the sheath 126. For example, the core first and second members 125 and 127 are concentric, with the core second member 127 taking the form of a tube inside which the core first member 125 is movable. However, other configurations of the core first and second members 125 and 127 are also within the scope of the invention, such as for example and non limitingly, two elongated parallel wires. The core first and second members 125 and 127 typically extend between the proximal and distal ends of the device 100 so that the core first and second members 125 and 127 can be longitudinally moved relative to each other as described below. To that effect, the proximal end of the device 100 includes a suitable actuator 200, as seen in FIG. 14. In FIG. 14, filaments 132, described in further details below, are omitted for clarity reasons. The actuator 200 allows moving the core first member 125 relative to the core second member 127 and moving the core 124 relative to the sheath 126. In some embodiments, this is achieved by controlling independently directly the relative position of each of the core first and second members 125 and 127 relative to the sheath 126, as in FIGS. 14 to 16E. In other embodiments, the core first and second members 125 and 127 are movable relative to each other, and the core 124 is movable, as a single unit, relative to a distal part of the sheath 126. In yet other embodiments, as described below, the core first member 125 and the sheath 126 are linked to each other so that the core second member 127 and the sheath 126 move longitudinally relative to each other in opposite directions at all time when the actuator 200 is used. Any other suitable actuator known in the medical field to achieve this functionality is usable. The core first and second members 125 and 127 may be circumferentially fixed (as in the device 100) or circumferentially movable relative to each other.

Referring for example to FIG. 12, the core first member 125 typically protrudes from the core second member 127. The core first member 125 may include a substantially atraumatic tip section 148 that facilitates insertion of the device 100 into the uterus or other body cavity in which the device is usable. The tip section 148 has a maximal diameter section 149 of a diameter that is slightly larger than the diameter of the core second member 127, and therefore acts as a mechanical stop to prevent full withdrawal of the core first member 125 into the core second member 127, and tapers gradually distally relative to the maximal diameter section 149. This configuration also allows sealing of the filaments 132 from the environment before and after sample collection has been made to reduce or eliminate contamination. In other embodiments, the tip section 148 may be withdrawn in the core second member 127. Proximally to the maximal diameter section 149, the tip section 148 may be half-sphere shaped, or have any other suitable shape. For example, the tip section 148 is made of thermoplastic urethane (TPU). The tip section 148 typically has a sufficient flexibility to guide the device 100 properly during insertion into the uterus or other body cavity in which the device 100 is used. The TPU material hardness can range from Shore A 40 to Shore A 100, among other possibilities. In some embodiments, the shape of the tip section 148 is such that it can easily go into the cervical canal 22 to expand it and allow the core second member 127 to be placed inside the uterus while minimizing pain. The tip section 148 can be made of other soft polymers such as LDPE, or silicone rubber, among others. In a specific and non-limiting embodiment, the tip section 148 is at most 3.5 mm in diameter at its largest point.

The core first member 125 may be hollow, similarly to the core 124, and provided with apertures (not shown in the drawings) for allowing collection of a fluid sample. In other embodiments, the core first member 125 may include a twisted-wire metal shaft that maintains the overall rigidity of the device 100. The metal is, for example, stainless steel 304 or stainless steel 316L, but other metals or other suitable materials are also usable. For example, and non-limitingly, the core first member 125 has a diameter of from about 1 mm to about 2 mm.

The core 124 further includes a plurality of elongated filaments 132. The filaments 132 are each mounted to the core first and second members 125 and 127 respectively at longitudinally spaced apart first and second mounting locations 133 and 135 therealong and each defining a filament detached section 137 between the first and second mounting locations 133 and 135, typically in end portions of the filaments 132, that is freely deformable and movable relative to the core first and second members 125 and 127. In other words, the filaments 132 are each mounted to both the core first and second members 125 and 127 with a portion thereof, the filament detached section 137, typically intermediate the ends of the filaments 132, movable relative to the core first and second members 125 and 127 and extending between the core first and second members 125 and 127. Moving the core first and second members 125 and 127 relative to each other will result in deformation of the filaments 132, and more specially of the filament detached sections 137.

The filaments 132 are for example distributed along the whole circumference of the core 124, but other distributions are within the scope of the invention. Typically, the filaments 132 are configured and have mechanical properties such that they buckle relatively easily when the first and second mounting locations 133 and 135 are brought closer to each other compared to a configuration in which the filaments 132 are such that the filament detached section 137 is parallel to the core 124. In a specific and non-limiting example, the filaments 132 are made of Nylon 66 and have a round cross-section, with a diameter of between 30 and 100 microns. However, other materials, dimensions and cross-sections, for example those recited with respect to the brush bristles 32, are within the scope of the invention. While only a few filaments are illustrated in FIG. 12, the number of filaments may be relatively large, for example 100 or more. In some embodiments, the filaments 132 have an oval cross-section, which may facilitate buckling when compared to round cross-sections for an equal cross-sectional area. The filaments 132 may also be hydrophilic to promote collection of fluids from the body cavity.

The core first and second members 125 and 127 are longitudinally movable between core stowage and collection configurations. The first and second mounting locations 133 and 135 are closer to each other in the core collection configuration than in the core stowage configuration.

Typically, in the core stowage configuration, the first and second mounting locations 133 and 135 are maximally spaced apart from each other. The corresponding distance between the first and second mounting locations 133 and 135 may be set by how much the filaments 132 can be extended, or can be limited by mechanical interferences in the actuator 200. In this latter case, the filament detached sections 137 may not be completely under tension, or the mechanical interference may be set to induce a predetermined tension in the filament detached sections 137.

In the core stowage configuration, the filament detached sections 137 may extend substantially longitudinally and may extend parallel to the core first member 125 or helically relative to the core first member 125, with a relatively large pitch. In the core collection configuration, the first and second mounting locations 133 and 135 are minimally spaced apart from each other. The minimal distance may be set by mechanical interference of the filaments 132 at the first and second mounting locations 133 and 135 when the latter get close to each other, or may be limited by mechanical interference in the actuator. In this latter case, the first and second mounting locations 133 and 135 may still be spaced apart from each other or may be adjacent to each other. Typically, this causes bulging of the filaments 132 relative to the core first and second members 125 and 127. In some embodiments, in the core collection configuration, the filaments 132 bulge to contact the uterine wall 16 so that cells can be collected thereby. In this configuration, the filaments 132 minimize tissue injury as there are no pointed ends interfacing with the tissue, only soft looped bundles of filaments 132. Also, intrauterine fluids may be collected in a relatively large amount as a major portion of the length of the filaments 132 interface with the tissue, not just the end tips.

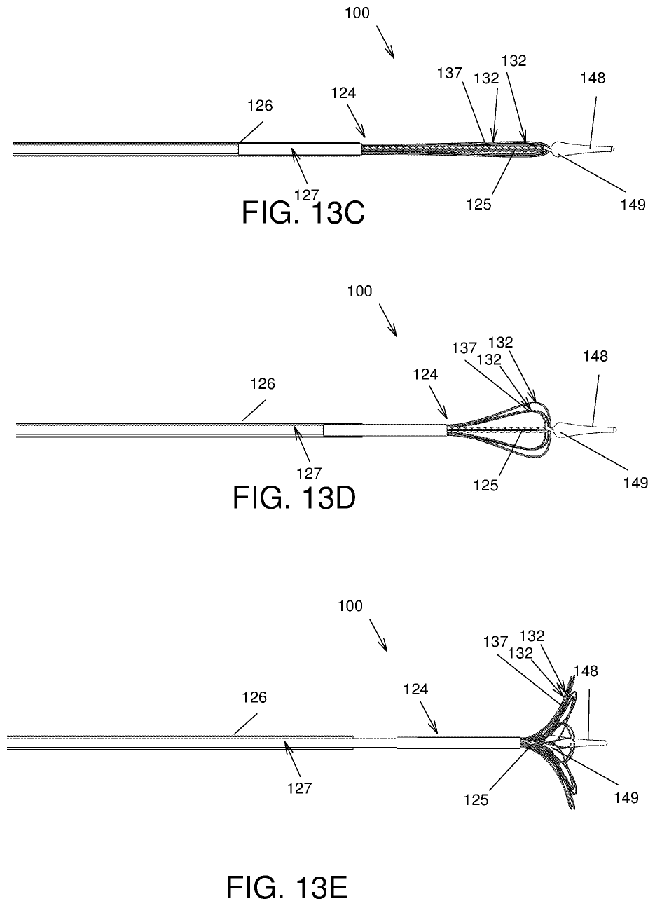

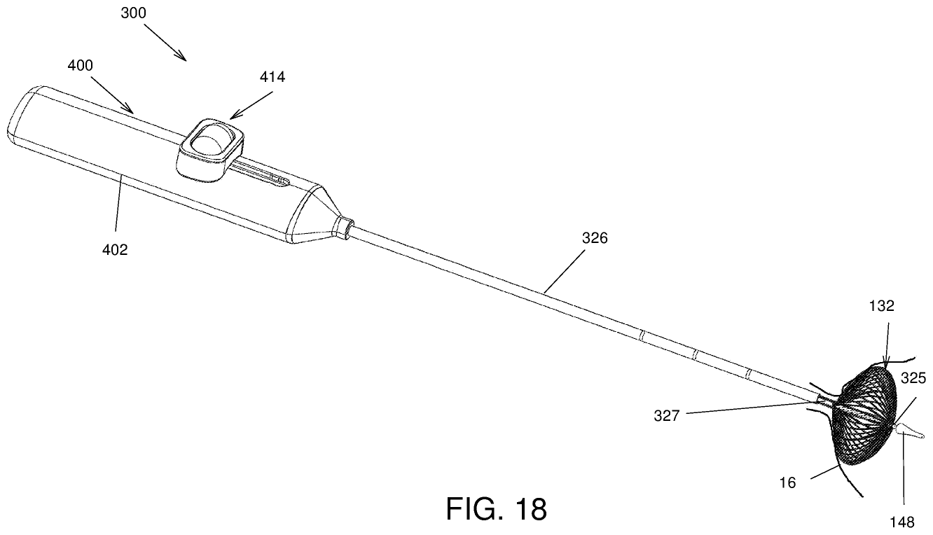

Thus, in some embodiments, in the core stowage configuration, the filament detached sections 137 all extend substantially longitudinally so as to span a stowage volume extending radially less than the sheath 126, and, in the core collection configuration, with the device 100 in the device expanded configuration, the filament detached sections 137 span a collection volume extending radially more than the sheath 126. The collection volume may take any suitable shape. For example, in the device 100 and as seen in FIG. 13E, the collection volume may be substantially tulip flower shaped. In another example, as seen in the device 300 shown in FIG. 18, the collection volume is substantially ellipsoidal. Also, in some embodiments the filament have an untensed configuration that is curved and/or are beaded, which may increase collection efficiency.

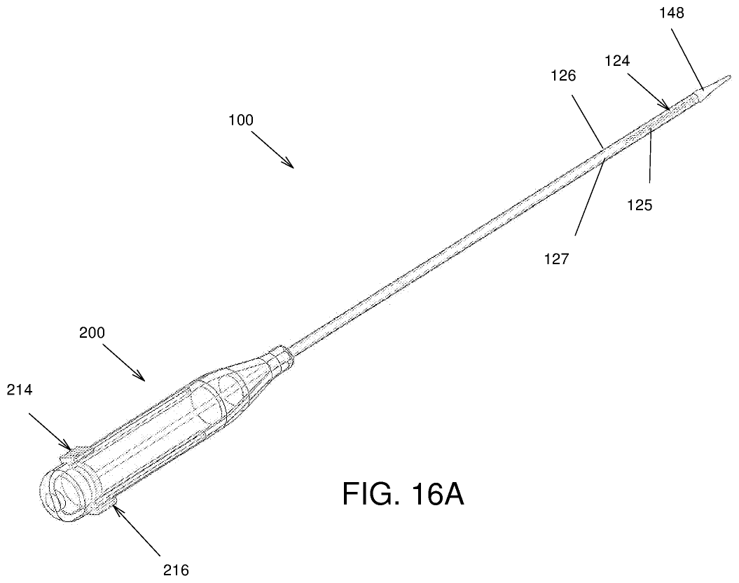

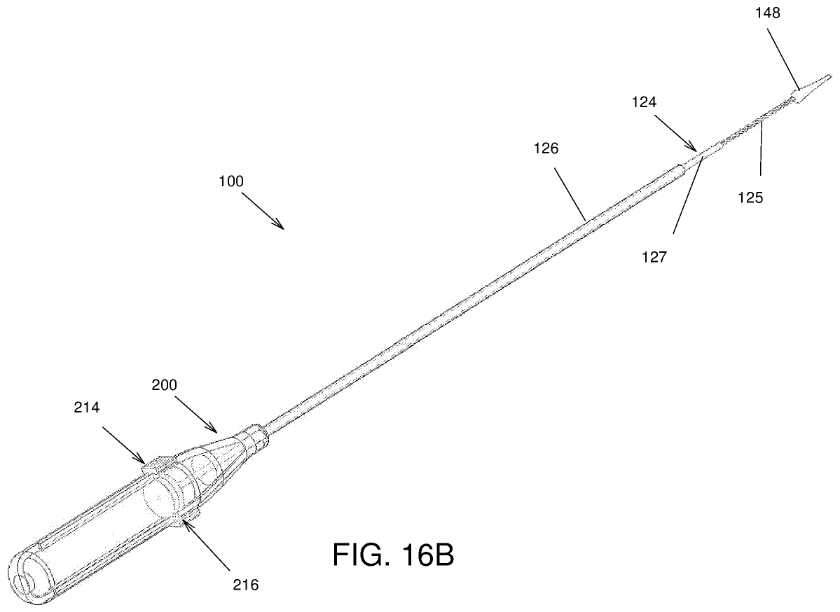

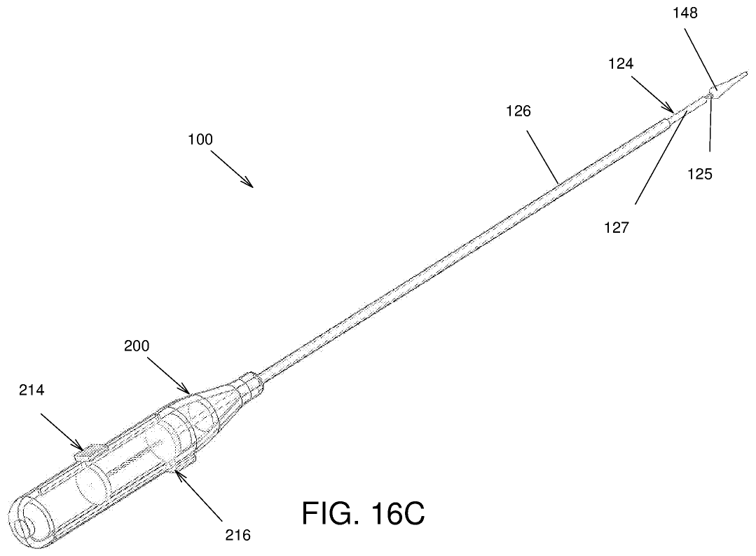

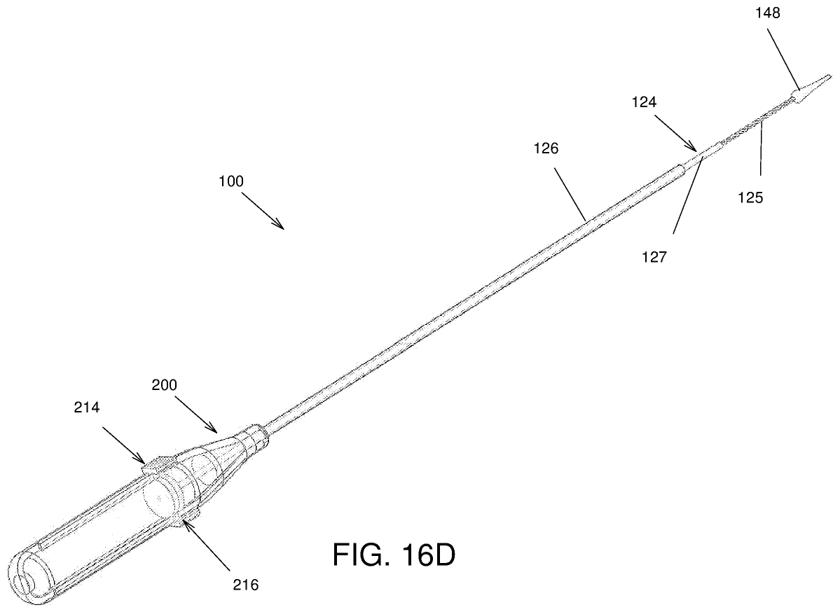

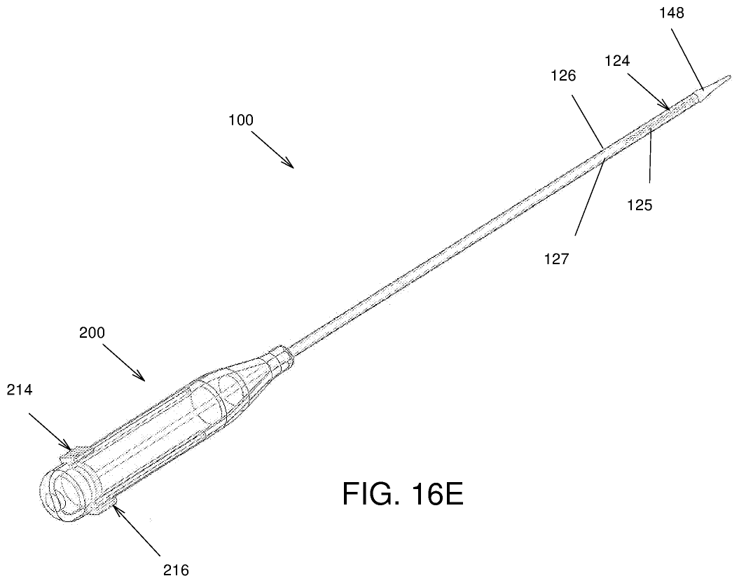

As in the device 10, the device 100 is configurable between a device retracted configuration (seen for example in FIG. 16A) and a device expanded configuration, seen for example in FIG. 16B). In the device retracted configuration, the filaments 132 are contained within the sheath 126, and, in the device expanded configuration, the at least part of the filament detached section 137 is distally outside the sheath 126.

In some embodiments, the filaments 132 are secured to the core first member 125 by twisting the filaments 132 simultaneously and jointly with metal wires while manufacturing the core first member 125 or adhering the filaments 132 to the core first member 125. Also, the filaments 132 may be secured to the core second member 127 through a core sleeve 129 covering a proximal end section of the filaments 132 and firmly maintaining the filaments 132 between a core second member main tube 131 and the core sleeve 129 provided at the distal end of the core second member main tube 131. For example, the core sleeve 129 is shrink wrapped, press fitted, adhesively secured or otherwise secured to the core second member main tube 131. Other suitable manners or securing the filaments 132 to the core first and second members 125 and 127 may also be used.

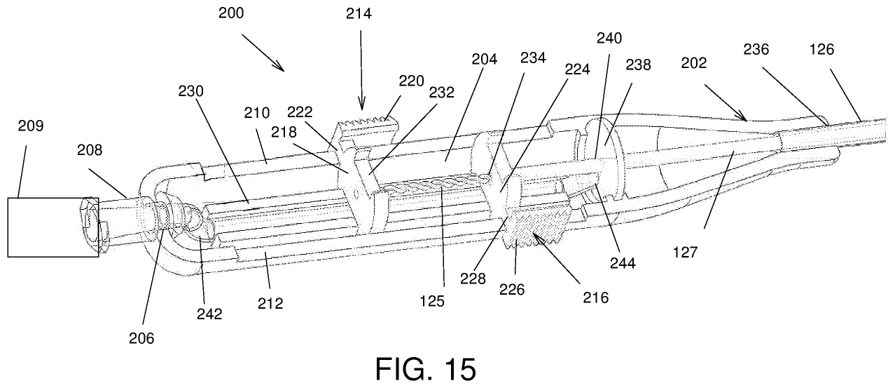

FIG. 15 better illustrates the actuator 200. The actuator 200 is usable for selectively configuring the device 100 between the device retracted and expanded configurations and selectively moving the core 124 between the core stowage and collection configurations. The actuator 200 includes an actuator body 202 defining an actuator body cavity 204. The actuator body 202 is for example generally cylindrical so as to be relatively easily held in the hand of an operator of the device 100, and may be rounded or tapered proximally and/or distally. Optionally, in some embodiments, a proximal aperture 206 leads into the actuator body cavity 204 and is provided with a Luer lock 208 to allow connection of a syringe 209 thereto so that the syringe in is fluid communication with the sheath 126 and allows exertion of an underpressure or an overpressure relative to atmospheric pressure at the sheath distal end 136 (seen in FIG. 12), by moving the plunger of the syringe. First and second lateral slits 210 and 212 each extending longitudinally along the actuator body 202 also lead into the actuator body cavity 204.

First and second sliders 214 and 216 are mounted to the actuator body 202 so as to be movable longitudinally therealong between respectively first slider proximalmost and distalmost positions and second slider proximalmost and distalmost positions. While the first and second sliders 214 and 216 are mounted in the actuator body cavity 204, alternative sliders are usable in alternative actuators that don't include a cavity. For example, and non-limitingly, such an actuator may take the form of a stem on which sliders are movable. Generally speaking, the first slider 214 is jointly movable with a first element selected from the core first and second members 125 and 127 and the sheath 126, the second slider 216 is jointly movable with a second element different from the first element and selected from the core first and second members 125 and 127 and the sheath 126, and a third element different from the first and second elements and selected from the core first and second members 125 and 127 and the sheath 126 is fixed relative to the actuator body. This allows achieving the core stowed and collection configurations and the device retracted and expanded configurations in many alternative manners.

In the actuator 200, the first and second sliders 214 and 216 are respectively operatively coupled to the core first and second members 125 and 127 so that moving the first and second sliders 214 and 216 relative to the actuator body 202 correspondingly translates the core first and second members 125 and 127 relative to the actuator body 202. In some embodiments, the sheath 126 is fixedly mounted to the actuator body 202, which causes movements of the first and second sliders 214 and 216 relative to the actuator body 202 to cause corresponding movements of the core first and second members 125 and 127 relative to the sheath 126.

The first slider 214 includes a first slider body 218 mounted in the actuator body cavity 204 so as to be movable longitudinally therealong, a first slider button 220 provided outside of the actuator body 202 and a first slider link 222 extending therebetween through the first lateral slit 210. Similarly, the second slider 216 includes a second slider body 224 mounted in the actuator body cavity 204 so as to be movable longitudinally therealong, a second slider button 226 provided outside of the actuator body 202 and a second slider link 228 extending therebetween through the second lateral slit 212. Thus, the first and second sliders 214 and 216 protrude from the actuator body 202 and are independently movable therealong. The first slider body 218 is proximal to the second slider body 224 in the actuator body cavity 204.

The first slider body 218 and the actuator body cavity 204 are typically complementarily shaped so that the former is constrained to move only longitudinally therealong, with little or no lateral movements. For example, the actuator body cavity 204 is substantially cylindrical and defines one or more guides 230, such as two guides 230, only one of which is shown in FIG. 15, extending longitudinally therealong and protruding inwardly thereinto. The lateral cross-sectional configuration of the actuator body cavity 204 is substantially constant along the whole path along which the first and second slider bodies 218 and 224 are movable. The first slider body 218 is for example substantially disc-shaped and defines one or more recesses 232 engaging and receiving the one or more guides 230. The core first member 125 is secured at its proximal end to the first slider body 218.

The second slider 216 is similar to the first slider 214, and is therefore not described in details herein. The core second member 127 is secured at its proximal end to the second slider body 224. A difference between the first and second sliders 214 and 216 resides in that the second slider body 224 defines a slider aperture 234 extending longitudinally therethrough and leading into the core second member 127. The core first member 125 extends through the slider aperture 234 and into the core second member 127.

The actuator body 202 also defines a body distal aperture 236 receiving the proximal end of the sheath 126. Typically, an hermetic seal is formed between the body distal aperture 236 and the sheath 126. The core first and second members 125 and 127 extend through the body distal aperture 236 and the sheath 126.

In some embodiments, a seal 238, for example a silicone membrane, provided with a seal first aperture 240 extends across the actuator body cavity 204 between the body distal aperture 236 and the second slider distalmost position achievable by the second slider 216. The core second member 127 extends through the seal first aperture. A tube 242 extends between the Luer lock 208 and a seal second aperture 244 provided in the seal 238. The tube may also extend in a recessed portion of one of the guides 230 so as to not interfere with movements of the first and second sliders 214 and 216. The seal 238 seals the portions of the actuator body cavity 204 proximal and distal to the seal 238 from each other. Therefore, any negative or positive pressure (relative to atmospheric pressure) exerted at the Luer lock 208, for example using a syringe, is transmitted to the interior of the sheath 126.

FIGS. 13A to 13E, paired with FIGS. 16A to 16E in which the filaments 132 are omitted, illustrate successive steps in example of use of the device 100. First, in FIG. 13A and 16A, the first and second sliders 214 and 216 are respectively in the first and second sliders proximalmost positions. In this configuration, only the distal tip 148 protrudes from the sheath 126 and the filament detached sections 137 are axially elongated and contained between the sheath 126 and the core first member 125.

Then, as see in FIG. 16B, the first and second sliders 214 and 216 are moved respectively to the first and second slider distalmost positions, followed by proximal withdrawal of the first slider 214 to a predetermined position intermediate the first slider proximalmost and distalmost positions, as seen in FIG. 16C, which may be a predetermined position identified by indicia on the actuator body 202. The filaments 132 then achieve a configuration similar to the configuration of FIGS. 13D or 13E. In alternative embodiments, as seen in the sequence of FIGS. 13B and 13C when an actuator similar to that of the device 10 is used, the device 100 is moved to a device retracted configuration, which moves the core 124 outside of the sheath 126 so that the filament detached sections are exposed outside of the sheath 126. Then, as seen in the sequence of FIGS. 13D and 13E, the core 124 is moved to the core collection configuration by moving the core first and second members 125 and 127 relative to each other, which causes the filaments 132 to bulge. In both types of devices, the filaments 132 may be used to collect a biological sample. If desired, a syringe may be used to collect through the sheath 126 fluids adjacent the distal end of the sheath 126. Such collection can be performed at any suitable moment during use of the device 100.

Withdrawal of the filaments 132 back into the sheath 126 then proceed by reversing the above-described steps. For example, the first slider 214 is pushed back to the first slider distalmost position, as seen in FIG. 16D, and the first and second sliders 214 and 216 are withdrawn back together to their proximalmost positions, as seen in FIG. 16E.

Generally speaking, the device 100 allows for a method for collecting a biological sample in a body cavity accessible through a body passageway narrower than the body cavity. In this method, first, with the filaments 132 contained in the sheath 126 with the filament ends spaces apart from each other, the sheath 126 is inserted in the body passageway so that the sheath distal end 136 is positioned adjacent or inside the body cavity. For example the sheath 126 is inserted in a vagina 12 until adjacent the cervical canal 22. Then, the filaments 132 are distally deployed outside the sheath 126 and filament end portions, for example at the first and second filament mounting locations 133 and 135 are moved substantially longitudinally towards each other to cause the filaments 132 to buckle in the filament intermediate portions, for example in the filament detached sections 137, and span a volume extending radially to a greater extent than the sheath 126. Subsequently, the biological sample is collected with the filaments intermediate portions and the filaments are withdrawn inside the sheath, followed by withdrawal of the sheath from the body passageway. In the proposed method, in some embodiments, collecting the biological sample includes moving the filament intermediate portions along a mucosa delimiting the body cavity with at least part of the filament detached sections 137 and the mucosa parallel to each other, for example by moving the filaments along the uterine wall. However, in other embodiments, some or most of the filaments don't contact the mucosa and only fluids in the body cavity are collected. In contrast to conventional collection methods used to collect biological samples with devices including bristles, the present device collects the biological sample without having the free end of bristles in contact with biological tissues. This provides a much gentler and less traumatic contact between the filaments 132 and the biological tissues.

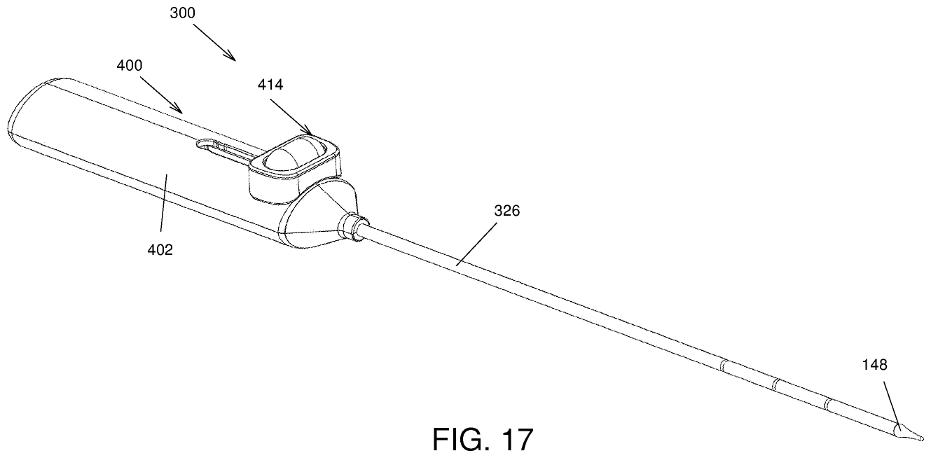

Referring to FIGS. 17 to 19, there is shown various aspects of yet another alternative device 300. The device 300, and its actuator 400, are similar to the device 100 and actuator 200 and only the differences therebetween are detailed below. To note, the locations of the first and second sliders 414 and 416 are opposed to those of the first and second sliders 214 and 216 as only the distalmost slider of the device 300 protrudes from the actuator 400. One difference between the devices 100 and 300 is that in the device 300, core configurations and device configurations are coupled to each other. More specifically, movements between the device retracted and expanded configurations and movements between the core stowage and collection configurations are linked such that when the device 300 in the device retracted configuration (as seen in FIG. 17), the core 324 is in the core stowage configuration, and when the device 300 is in the device expanded configuration (as seen in FIG. 18), the core 324 is in the core collection configuration.

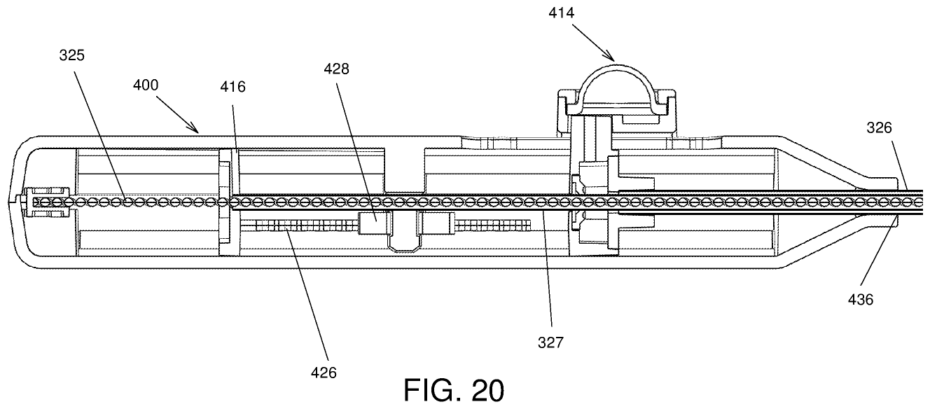

Structurally, this is achieved in the device 300 by having only one slider, for example the first slider 414 protruding from the actuator body 402. The other slider, for example the second slider 416 is enclosed in the actuator body 402. However, the first and second sliders 414 and 416 are operatively coupled to each other so that proximally directed and distally directed movements of the first slider 414 occur jointly and simultaneously with respectively distally and proximally directed movements of the second slider 416. In other words, the first and second sliders 414 and 416 move in a reciprocating movement relative to each other. Referring to FIG. 20, the sheath 326 is movable jointly with the first slider 414, by being secured thereto. The sheath 326 is therefore mounted through the body distal aperture 436 so as to be slidable therethrough. The core second member 327 is movable jointly with the second slider 416, by being secured thereto. The core first member 325 is longitudinally fixed relative to the actuator body 402, for example by being secured to the proximal end thereof, the first and second sliders 414 and 416 being configured to allow movements thereof freely relative to the core first member 325, for example by defining through apertures allowing free passage of the core first member 325 therethrough. In some embodiments (not shown in the drawings), the sheath 326 is also in fluid communication with a passageway extending through the first slider 414 and allowing connection of a syringe thereto, similarly to the syringe attachment of the device 100.

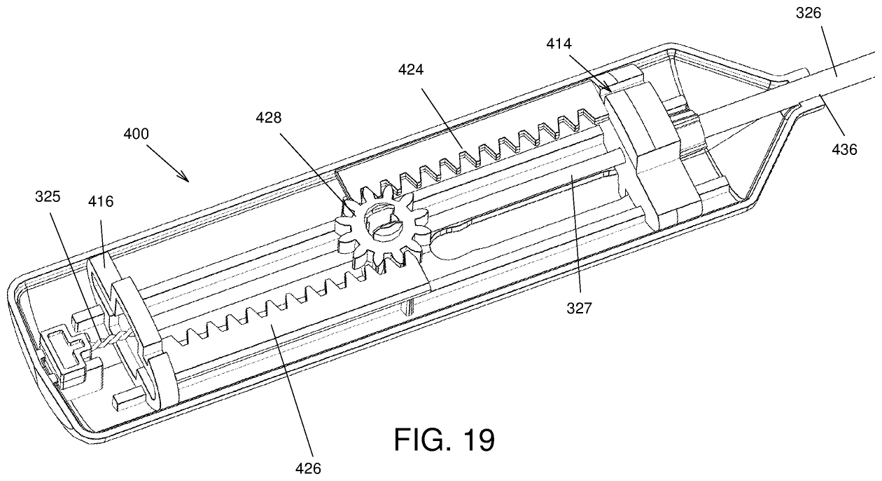

As better seen in FIG. 19, the first and second sliders 414 and 416 include respectively first and second toothed racks 424 and 426 oriented substantially longitudinally. A toothed pinion 428 extends between the first and second toothed racks 428 and couples movements of the first and second toothed racks 424 and 426 to each other so that the first and second toothed racks 424 and 426, and consequently the first and second sliders 414 and 416, move in opposite directions relative to each other. For example, the first and second toothed racks 424 and 426 are provided on laterally opposite sides of the toothed pinion 428 and the first and second sliders 414 and 416 each configured to allow free longitudinal movements of the first and second toothed racks relative to each other.

In other embodiments (not shown in the drawings), the first and second sliders 414 and 416 are completely enclosed in the actuator body 402 and it is the toothed pinion 428 that is actuated through a knob extending axially therefrom and provided outside of the actuator body 420.

FIG. 18 illustrates schematically how the filaments 132 contact tangentially an organ from which the biological sample is to be collected, here exemplified as the uterine wall 16. The ends of the filaments 132 are secured to the core first and second members 325 and 327. In some embodiments, the ends of the filaments 132 are completely enclosed in the structure of the devices 100 or 300 so as not to protrude in the body cavity in which the sample is collected. Thus, only portions of the filaments 132 away from such ends may contact the uterine wall. Furthermore, the filaments, when deployed for collection, have a generally curved shape that can be relatively easily deformed to conform to the shape of a portion or the whole uterine cavity, for example around a whole circumference thereof, and to also be relatively easily deflected if any pressure is exerted on the uterine cavity, so as to exert only relatively small pressures and forces on the uterine wall during sample collection.

In empirical tests, embodiments of the device 10 (referred to as “DEV1”) and of the device 100 (referred to as “DEV2”) were used to collect a uterine fluid analog (was 99% glycerin at 20° C.) in a graduated cylinder. They were compared to a commercial device (TAO (TM) brush), which has a length of about 3-3.5 cm and a diameter of 9 French with relatively stiff bristles as this brush is a biopsy brush. Ten measurements were taken with each test article. Collection was performed by inserting the test article at a 0-degree angle (vertical) into the graduated cylinder, immediately followed by 5 full cycles of counter-clockwise rotation by smoothly rotating the handle of each device between two fingers; after which the device was removed, and the sample transferred to a vial containing 5 mL of methanol-based preservation solution (TP) (Preservcyt®, ThinPrep Pap Test, Hologic, Inc. Marlborough, Mass.), after which aliquots of each sample were transferred to a 96-well microplate, as shown in FIG. 4-10 (right), and analyzed in a TECAN SPARK M10 spectrophotometer for a quantitative measurement of the sample transferred to the preservation vial. In simplified volume collection tests, the control device (TAO™ Brush), showed a mean collection volume of 600 μL±204 μL. The liquid biopsy devices DEV1 and DEV2 collected 643 μL±370 μL and 774 μL±151 μL, respectively. The mean transferred volumes were 320 μL±207 μL, 424 μL±347 μL, and 436 μL±160 μL for the control, collector DEV1 and collector DEV2 respectively. The designed collectors show an increase of 131% and 135% of transferred sample with respect to the control.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can be modified, without departing from the spirit and nature of the subject invention as defined in the appended claims.

1. A device for collecting a biological sample, the device comprising:
a substantially elongated core including a core first member and a tip section, the core further including a plurality of elongated filaments between the core first member and the tip section, the filaments each defining longitudinally spaced apart first and second mounting locations, the filaments being mounted to the core first member and the tip section respectively at the first and second mounting locations, the filaments each defining a filament detached section between the first and second mounting locations, the filament detached section being deformable and movable relative to the core first member and the tip sections; and
a substantially elongated sheath receiving at least part of the core thereinto, the sheath defining longitudinally opposed sheath proximal and distal ends, the sheath and the core being longitudinally movable relative to each other;
the device being configurable between a retracted configuration and a collection configuration, wherein, in the retracted configuration, the filament detached sections are contained within the sheath, and, in the collection configuration, at least part of each of the filament detached sections is distally outside the sheath;
the core first member and the tip section being longitudinally movable relative to each other between the retracted configuration and the collection configuration, wherein the first and second mounting locations are closer to each other in the collection configuration than in the retracted configuration.

2. The device as defined in claim 1, wherein, in the retracted configuration, the filament detached sections extend generally longitudinally so as to span a retracted volume extending radially less than the sheath, and, in the collection configuration the filament detached sections span a collection volume extending radially more than the sheath.

3. The device as defined in claim 2, wherein the collection volume is substantially ellipsoidal.

4. The device as defined in claim 2, wherein the collection volume is substantially tulip flower shaped.

5. The device as defined in claim 1, further comprising an actuator for selectively configuring the device between the retracted and collection configurations.

6. (canceled)

7. (canceled)

8. (canceled)

9. The device as defined in claim 5, wherein the actuator defines an actuator body and at least one slider movable longitudinally along the actuator body, the slider being jointly movable with a first element selected from the core first member and the sheath.

10. The device as defined in claim 9, wherein the actuator body is hollow and the at least one slider is mounted in the actuator body so as to be movable longitudinally relative thereto.

11. The device as defined in claim 10, wherein the at least one slider protrudes from the actuator body and is independently movable therealong.

12. The device as defined in claim 10, wherein the sheath is movable jointly with the at least one slider, and the core first member is longitudinally fixed relative to the actuator body.

13. (canceled)

14. The device as defined in claim 1, wherein the sheath is in fluid communication with a syringe for allowing exertion of an underpressure or an overpressure relative to atmospheric pressure at the sheath distal end.

15. The device as defined in claim wherein the filaments are hydrophilic.

16. The device as defined in claim 1, wherein at the core first member and/or the sheath is hollow, in fluid communication with a vacuum source and provided with at least one aperture leading thereinto and provided adjacent the filaments, whereby exerting a vacuum using the vacuum source collects fluids adjacent the filaments through the at least one aperture.

17. A method for collecting a biological sample in a body cavity accessible through a body passageway narrower than the body cavity, the method comprising:
using a sample collection device having an elongated tubular sheath defining a sheath distal end and a plurality of filaments mounted to an elongated core member, the plurality of filaments defining opposed filament end portions and filament intermediate portions extending therebetween;
with the filaments contained in the sheath with the filament end portions longitudinally spaced apart from each other, inserting the sheath in the body passageway so that the sheath distal end is positioned adjacent to or inside the body cavity;
distally deploying the filaments outside the sheath in the body cavity by moving the sheath and the filaments relative to each other the filament end portions moving substantially longitudinally towards each other to cause the filament intermediate portions to expand radially outwardly and span a volume extending radially to a greater extent than the sheath;
collecting the biological sample with the filaments intermediate portions;
withdrawing the filaments inside the sheath; and
withdrawing the sheath from the body passageway.

18. The method as defined in claim 17, wherein the biological sample includes biological fluids.

19. The method as defined in claim 17, wherein the biological sample includes cells.

20. The method as defined in claim 17, wherein collecting the biological sample includes moving the filament intermediate portions along a mucosa delimiting the body cavity with at least part of the filament intermediate portion and the mucosa parallel to each other.

21. The method as defined in claim 17, wherein the body cavity is a uterine cavity defined by an uterine wall, collecting the biological sample including moving the filament intermediate portions tangentially along the uterine wall.

22. The method as defined in claim 17, wherein distally deploying the filaments includes using an actuator of the sample collection device to move the sheath and the filaments relative to each other.

23. The method as defined in claim 22, wherein the actuator includes an actuator body and a slider, the method further comprising moving the slider longitudinally along the actuator body, the sheath being jointly movable with the slider to distally deploy the filaments.

24. The method as defined in claim 17, further comprising using a syringe fluidly coupled to the sample collection device to exert a vacuum and collect the biological sample from the plurality of filaments.

25. A device for collecting a biological sample, the device comprising:
a core that is substantially elongated and includes a sample collector at a distal end thereof;
a substantially elongated sheath receiving at least part of the core therein, the sheath defining opposed sheath proximal and distal ends axially spaced apart along a longitudinal axis, the sheath and the core being displaceable relative to each other along the longitudinal axis between a retracted configuration and a collection configuration, wherein, in the retracted configuration, the sample collector is contained within the sheath, and, in the collection configuration, at least part of the sample collector is exposed distally outside the sheath; and
an actuator at a proximal end of the device, the actuator including an actuator body and a slider mounted at least partially in the actuator body and movable longitudinally relative thereto by a user, the slider being operatively coupled to the sheath and/or the core to cause the relative displacement thereof between the retracted configuration and the collection configuration.

26. The device as defined in claim 25, wherein the sample collector includes a plurality of elongated filaments, and the sheath is jointly movable with the slider, wherein, in the retracted configuration, the plurality of elongated filaments are compressively contained within the sheath in a compressed configuration, and, in the collection configuration, the plurality of elongated filaments are outside the sheath, distally to the sheath distal end, in an expanded configuration adapted for sample collection wherein the plurality of elongated filaments occupy a larger volume than in the compressed configuration.

27. The device as defined in claim 26, wherein the actuator body is hollow, the slider is mounted in the actuator body, protrudes from the actuator body and includes a slider button for actuation of the slider.

28. The device as defined in claim 25, wherein the core includes a core first member and a tip section, the sample collector located between the core first member and the tip section, the core first member and tip section being longitudinally movable relative to each other, the sample collector including a plurality of elongated filaments each defining longitudinally spaced apart first and second mounting locations, each filament being mounted to the core first member and the tip section respectively at the first and second mounting locations, each of the plurality of filaments defining a filament detached section between the first and second mounting locations, the filament detached section being deformable and movable relative to the core first member and tip section, the sheath and the core first member and the tip section being longitudinally movable relative to each other, wherein, in the retracted configuration, the filament detached sections are contained within the sheath, and, in the collection configuration, at least part of each of the filament detached sections is distally outside the sheath, and wherein the core first member and the tip section being longitudinally movable relative to each other between retracted and collection configurations, wherein the first and second mounting locations are closer to each other in the collection configuration than in the retracted configuration.

29. The device as defined in claim 28, wherein the actuator body is hollow, the slider is mounted in and protrudes from the actuator body and is independently movable therealong, the slider jointly movable with one of the core and the sheath, and the other of the core and the sheath is fixed relative to the actuator body.

30. The device as defined in claim 29, wherein the slider includes a slider body mounted inside the actuator body, a slider button disposed outside of the actuator body, and a slider link extending therebetween through a first lateral slit in the actuator body.

31. The device as defined in claim 25, wherein the actuator body includes a syringe connector at a proximal end, the syringe connector defining a proximal aperture leading into the actuator body and in fluid communication with the sheath and/or the core.

32. A device for collecting a biological sample, comprising:
an elongated body having a core and a protective sheath, the core and protective sheath being longitudinally displaceable relative to each other, the core including a sample collector at a distal end thereof; and
an actuator mounted to a proximal end of the elongated body, the actuator including an actuator body and slider movable longitudinally relative to the actuator body, the slider being jointly movable with one of the core and the protective sheath, the other of the core and the protective sheath being fixed relative to the actuator body.

33. The device as defined in claim 32, wherein the actuator body is hollow, the slider is mounted in the actuator body and protrudes from the actuator body, the slider includes a slider body mounted inside the actuator body, a slider button disposed outside of the actuator body, and a slider link extending therebetween through a lateral slit in the actuator body.

34. The device as defined in claim 33, wherein the slider body and the actuator body are complementarily shaped so that the slider body is operable to only move longitudinally along the actuator body with little or no lateral movements therealong.

35. The device as defined in claim 32, wherein the protective sheath receives at least part of the core therein, the core including a core first member and a tip portion, the sample collector including a plurality of filaments between the core first member and the tip portion, each of the plurality of filaments defining longitudinally spaced apart first and second mounting locations respectively mounted to the core first member and the tip portion, each of the plurality of filaments defining a filament detached section between the first and second mounting locations, the filament detached section being deformable and movable relative to the core first member and tip portion.

36. The device as defined in claim 35, wherein the actuator is operable for selectively configuring the device between a retracted configuration and a collection configuration, wherein, in the retracted configuration, the filament detached sections are contained within the protective sheath, and, in the collection configuration, at least part of each of the filament detached sections is distally outside the protective sheath, and wherein the first and second mounting locations are closer to each other in the collection configuration than in the retracted configuration.

37. The device as defined in claim 35, wherein the actuator body defines a body distal aperture receiving the proximal end of the protective sheath, the core first member extending through the body distal aperture and the protective sheath.

38. The device as defined in claim 35, wherein the core defines a core outer surface and a distal brush section, the brush section provided with brush bristles extending from the core outer surface, the protective sheath receiving at least part of the core thereinto, the protective sheath defining axially opposed sheath proximal and distal ends.

39. The device as defined in claim 38, wherein the actuator is operable to configure the device between a retracted configuration and a collection configuration, wherein, in the retracted configuration, the brush bristles are compressively contained within the protective sheath in a brush bristle compressed configuration, and, in the collection configuration, the brush bristles are outside the protective sheath, distally to the sheath distal end, in a brush bristle expanded configuration wherein the brush bristles occupy a larger volume than in brush bristle compressed configuration.

40. The device as defined in claim 32, wherein the actuator body includes a syringe connector at a proximal end, the syringe connector defining a proximal aperture leading into the actuator body and in fluid communication with the sheath and/or the core.
